(19) United States
(12) Patent Application Publication
(10) Pub. No.: US 2002/0019670 A1
(43) Pub. Date: Feb. 14, 2002

(54) IMPLANTABLE TISSUE AUGMENTATION DEVICE

(76) Inventors: JERALD M. CRAWLEY, FLAGSTAFFF, AZ (US); COREY W. DIETRICH, FLAGSTAFF, AZ (US); PETER R. GIOVALE, FLAGSTAFF, AZ (US); THOMAS M. O'HARA, FLAGSTAFF, AZ (US); ROBERT J. VONSEGGERN, FLAGSTAFF, AZ (US)

Correspondence Address: WAYNE D HOUSE, W L GORE AND ASSOCIATES INC, 551 PAPER MILL ROAD, PO BOX 9206, NEWARK, DE, 197149206

(21) Appl. No.: 08/808,291

(22) Filed: Feb. 28, 1997

(51) Int. Cl. A61F 2/02

(52) U.S. Cl. 623/11.11; 623/13.2; 623/15.12

(57) ABSTRACT

An implantable tissue augmentation device having a multiplicity of biocompatible strands, each of said strands having at least one end wherein the multiplicity of strands are integrally joined at the at least one end of each of the multiplicity of strands. Optionally, the augmentation device has an attachment feature allowing easy attachment to a suture, a needle or other surgical instrument. The strands can have various cross sectional configurations such as rectangles or polygons.

### FIELD OF THE INVENTION

The present invention relates to cosmetic augmentation devices, their manufacture and use to augment or fill wrinkles in tissue. More specifically the present invention relates to a multi-segmented array of biocompatible strands used to augment or correct tissue wrinkles, in particular facial wrinkles.

### BACKGROUND OF THE INVENTION

Tissue irregularities which include lip contour irregularities, nasolabial folds and perioral wrinkles, are commonly aesthetically treated by relatively noninvasive subcutaneous augmentation techniques. These subcutaneous, or under the skin (below the dermis) augmentation techniques rely on a biocompatible prosthesis being placed underneath the skin to fill out or augment the wrinkle. The prosthesis materials used for these augmentation procedures have included paraffin, liquid silicones, bovine collagens, sutures made from various materials and most recently porous expanded polytetrafluoroethylene (hereinafter ePTFE). The use of ePTFE in these subcutaneous augmentation procedures has produced favorable results since the ePTFE is easy to handle, supple, causes minimal tissue reactions and can be removed if required. The use of ePTFE in facial subcutaneous augmentation is widely published. Two recent publications include those of Cisneros et al. (1993) and Schoenrock, (1993). Cisneros, J.,Singla, R., Intradermal Augmentation with Expanded Polytetrafluoroethylene (Gore-Tex) for Facial Lines and Wrinkles, Clinical center of Laser and Cosmetic Surgery, Barcelona Spain, describes the use of ePTFE sutures, threaded below the dermis, by use of a needle. In this procedure, dermal incisions are made at both ends of the wrinkle and a needle or other tunneling tool is used to create a void or passageway below the dermis. A needle is then used to pull the prosthesis material through the incision into the void below the dermis, the ends of the filling material are trimmed to length and positioned under the skin, the needle is removed and the incisions are closed. By adding or subtracting strands of suture prior to closure, the wrinkle or crease can be optimally filled, thereby correcting the cosmetic irregularity. A similar procedure is described by Schoenrock, L., Reppucci, A., Gore-Tex in Facial Plastic Surgery, International Journal of Aesthetic and Restorative Surgery, Volume 1, Number 1, 1993. In this publication the use of ePTFE is similarly described in a subcutaneous filling procedure used for the augmentation of facial irregularities, wherein ePTFE sheets are cut into thin strips and pulled below the dermis by use of a needle. Also described is the use of ePTFE suture material for the filling of dermal voids. A publication by Waldman (1991) describes a method of filling deep or large dermal voids. Waldman, S., Randolph, Gore-Tex for Augmentation of the Nasal Dorsum: A Preliminary Report, Annals of Plastic Surgery, Volume 26, Number 6. June 1991, describes cutting strips of material from a biocompatible sheet, and for deep dermal voids, suturing two or three of these strips into a sandwich configuration. This layered and sutured device is then pulled below the dermis to fill the void.

These prior art procedures and devices have numerous drawbacks and disadvantages. The use of ePTFE suture as a prosthesis for filling below the dermis voids is very time consuming and cumbersome. The sutures must have the needles removed and discarded, and the suture must then be threaded into the eye of the surgical needle. In addition the suture strands must be aligned together to insure against short lengths, during which time excessive care is taken to not unthread the needle. If this suture bundle is tied to the needle, a large knot may be required, which must then be pulled through the facial incisions. Similarly, if the suture bundle is not tied, the diameter of the bundle adjacent to the needle is larger than the diameter of the remaining bundle. Once pulled through the incisions, the stranded sutures are often of unequal lengths, requiring excessive time to trim the strands to a uniform length. The suture is only available in limited diameters or thicknesses, which require a multitude of smaller sutures to fill a substantial void, further adding to the complexity of the surgical preparation and procedure. In addition the suture is only available in circular cross sections which may not optimally fill a given void. Any increase occurred in the handling or preparation of the suture bundle prior to or during the surgical procedure increases the risk of contaminating the prosthesis. Contamination of the prosthesis can result in chronic inflammation, increased foreign body reactions or extrusion of the prosthesis. Since these procedures are used to correct facial irregularities, any additional cosmetic deficiency, as a result of the surgery, is highly undesirable.

Thus a need exists for a subdermal augmentation prosthesis that overcomes the drawbacks and disadvantages of the prior art. A need exists for a multiply stranded prosthesis that can be rapidly prepared or attached to a threading needle, or is provided pre-attached to the needle, thereby minimizing handling and surgical time and reducing the potential for contamination. A need exists for a subdermal augmentation prosthesis that may be attached to a suture, a needle or other surgical tool without creating a bulge or other significant increase in diameter or transverse cross sectional area which will increase the difficulty of pulling the prosthesis into place below the dermis. A need also exists for a facial augmentation prosthesis which is of a highly biocompatible material, so as to minimize the inflammation response. In addition, a need exists for a facial augmentation prosthesis that is available in a variety of cross sectional shapes, and is available in various thicknesses and widths, exceeding those cross sectional areas available in conventional suture material. This will allow substantial filling of subdermal voids while minimizing the number of required strands. The present invention meets these needs.

### SUMMARY OF THE INVENTION

The present invention provides a subcutaneous augmentation prosthesis for the correction of tissue wrinkles that overcomes the drawbacks and disadvantages of the prior art. The devices of the present invention rely on a biocompatible material, pre cut into an array of a multiplicity of strands, with a strand having at least one end integrally joined to one end of at least two other strands. In addition the device of the present invention may be provided with an integral attachment feature that provides rapid and convenient attachment of a suture, needle or other surgical instrument. The attachment feature does not result in an increase in the diameter or transverse cross sectional area of the collective group of strands, thereby minimizing the resistance involved in pulling the prosthesis into place and minimizing the associated trauma. The devices of the present invention can be provided in various widths or thicknesses, with varying number of strands and with various strand shapes. The strands can be substantially rectangular in cross section, or have other cross sections such as triangles or polygons. Since the strands are integrally attached to each other on at least one end, the strands can be pulled smoothly through the incisions and under the dermis as a group or bundle with very little resistance or trauma in comparison to a group of strands tied together using a knot or other technique which increases the transverse cross sectional area of the device at that location and therefore increases the resistance to pulling. At the second incision, the joined region of the strands can be cut off, leaving individual strands in place under the skin. Individual strands can then be removed to optimally fill the subdermal void.

The integrally joined multiplicity of strands describes a multiplicity of strands joined together at one end of each of the at least three strands in integral, continuous fashion such that there is no significant discontinuity at the join such as would exist if the join was the result of a knot or some other joining means wherein the joined ends of the strands are discrete, distinct and readily identifiable as such by the naked eye and wherein the joined region has a larger transverse cross sectional area than the combined transverse cross sectional areas of the individual strands. Preferably, the integrally joined strands are the result of cutting a single piece of material into a multiplicity of strands while leaving the strands joined together at one end with the result that the joined ends of the strands are integral and continuous with each other and without readily identifiable disruption or discontinuity. Preferably, the region of the joined strand ends thus does not result in a significant (i.e., ten percent) increase in transverse cross sectional area beyond that of the collective individual strands. In an alternative embodiment, the integrally joined strands may be the result of joining by welding or adhesive whereby the join does not result in any increase in transverse cross sectional area beyond that of the collective individual strands.

The present invention provides a below-the-dermis augmentation prosthesis that is most preferably comprised of ePTFE. Preferred ePTFE materials are GORE-TEX® Subcutaneous Augmentation Material, GORE-TEX® Soft Tissue Patch, and GORETEX® Cardiovascular Patch, all available from W. L. Gore & Associates (Flagstaff, Ariz.). Alternatively, other biocompatible materials may be used including collagen, polypropylenes, polydimethyl siloxanes, polyethylenes (particularly polyethylene terephthalate), polyesters, polyurethanes, fluoropolymers (including PTFE fluorinated ethylene propylene and perfluorinated alkoxy resin) and various resorbable polymers such as polyglycolic acid, polylactic acid and copolymers thereof.

In an additional embodiment, the present invention provides an array of strands having a needle attachment feature in the integrally joined region that allows rapid attachment of the array to a suture, surgical needle or other surgical instrument.

In a further embodiment, the present invention provides an array of integrally joined strands having an integral needle attachment feature, so that when attached to a needle, the cross sectional area of the integrally joined region adjacent to the end of the needle, is of equal to or smaller than that of the collective individual strands.

In yet another embodiment, the present invention provides an array of strands that have various strand widths.

Another embodiment of the present invention provides an array of strands that have various strand shapes.

In an additional embodiment, the present invention provides an array of strands that have various strand thicknesses.

In another embodiment, the present invention provides an array of strands that are of a substantially rectangular cross section.

In a further embodiment, the present invention provides an array of strands that have polygonal cross sections.

In an additional embodiment, the present invention provides an array of strands formed from a hollow tube (such as a porous PTFE vascular graft), or an array of strands formed from a solid rod. These devices will conform to a substantially circular cross section when inserted below the dermis. These devices can optionally have attachment features for surgical needles or other surgical instruments.

In an additional embodiment the present invention provides an integral needle. This needle can be formed out of the same preferred porous strand material by densifying one end of the porous material using heat and/or pressure to substantially reduce or eliminate the porosity, thereby stiffening the one end to make it adequately rigid for use as a needle. Alternatively, the needle can be formed by combining one end of the porous strand material with an additional polymer such as fluorinated ethylene propylene in order to reduce the porosity and stiffen the one end.

Accordingly, the present invention is directed to an implantable tissue augmentation device comprising a multiplicity of strands of biocompatible material, each of said strands having at least one end wherein the multiplicity of strands are integrally joined at the at least one end of each of the multiplicity of strands.

These and other aspects and advantages will become more apparent when considered with the following detailed description, drawings and appended claims.

### BRIEF DESCRIPTION OF THE DRAWINGS

FIG. 1 shows a top view of a sheet of biocompatible material with various cut patterns which result in an array of integrally joined strands.

FIG. 2A shows a top view of an array of a multiplicity of strands, integrally joined at one end of each of the multiplicity of strands and including an eyelet in the joined region which provides for attachment to a suture, needle or other surgical instrument.

FIG. 2B shows a top view describing how the device may have a diameter less than or equal to that of an attached needle.

FIG. 3 shows a top view of an array of strands integrally joined at both ends of the strands. Also shown are attachment eyelets on both ends of the strands in the joined regions.

FIG. 4 shows a top view of an array of integrally joined strands connected together at one end and provided with an integral needle in the joined region.

FIG. 5 shows a top view of an array of integrally joined strands connected together at both ends. One end is provided with an eyelet while the other end is provided with an integrally joined suture.

FIG. 6 shows a top view of an array of strands integrally joined at one end wherein the individual strands are of different widths.

FIG. 7 shows a top view of an array of strands integrally joined at one end, wherein the individual strands are of varying widths, shapes and various end shape configurations.

FIG. 8 shows a top view of an array of strands integrally joined at one end, wherein the individual strands are of varying widths and shapes.

FIG. 9 shows a top view of an array of strands, cross connected to each other at different locations along their lengths and also integrally joined at one end.

FIGS. 10A-H show a perspective end view of the strands indicating a variety of possible transverse cross sectional shapes.

FIG. 11 shows an alternate configuration of the strands wherein the strands are braided together.

FIG. 12 shows a top view of an array of strands that are integrally joined in the center of the length of the array.

FIGS. 13A-D show perspective and end views of various integrally joined strand arrays, formed from hollow tubes and from solid rods

FIG. 14 shows a perspective view of an integrally joined array of strands, formed from a hollow tube or solid rod, and having an integral attachment feature (e.g. an eyelet) for use with a suture, needle or surgical instrument.

### DETAILED DESCRIPTION

The invention will now be described by reference to the figures and non-limiting embodiments.

The strands comprising the article of the present invention may be provided in various dimensions. For example, the overall length of the array of strands can have a range of 10 to 500 mm, with a preferred overall length of 50 to 200 mm, with a particularly preferred overall length of 125 mm. The overall width of the array can have a range of 4 to 30 mm, with a preferred overall width of 5 to 25 mm, with a particularly preferred overall width of 15 mm. The individual strand width of the array can have a range of 0.5 to 2.5 mm, with a preferred individual strand width of 0.5 to 1.5 mm, with a particularly preferred individual strand width of 1.0 mm. The overall thickness of the array can have a range of 0.1 to 5.0 mm, with a preferred overall thickness of 0.5 to 2.0 mm, with a particularly preferred overall thickness of 1.25 mm.

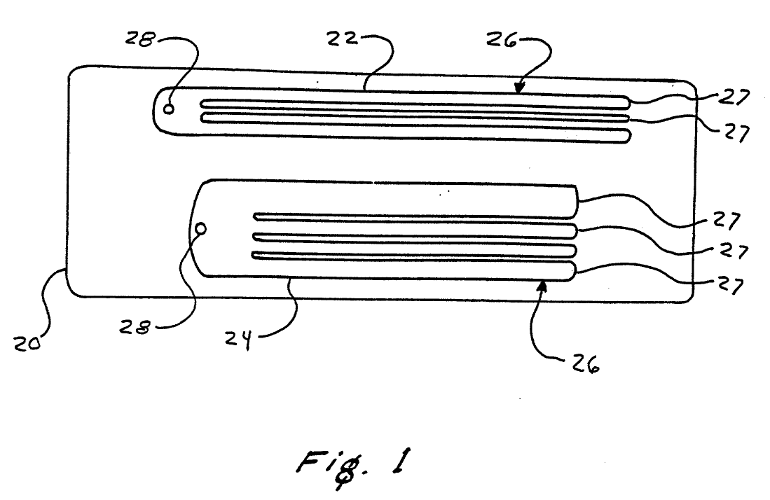

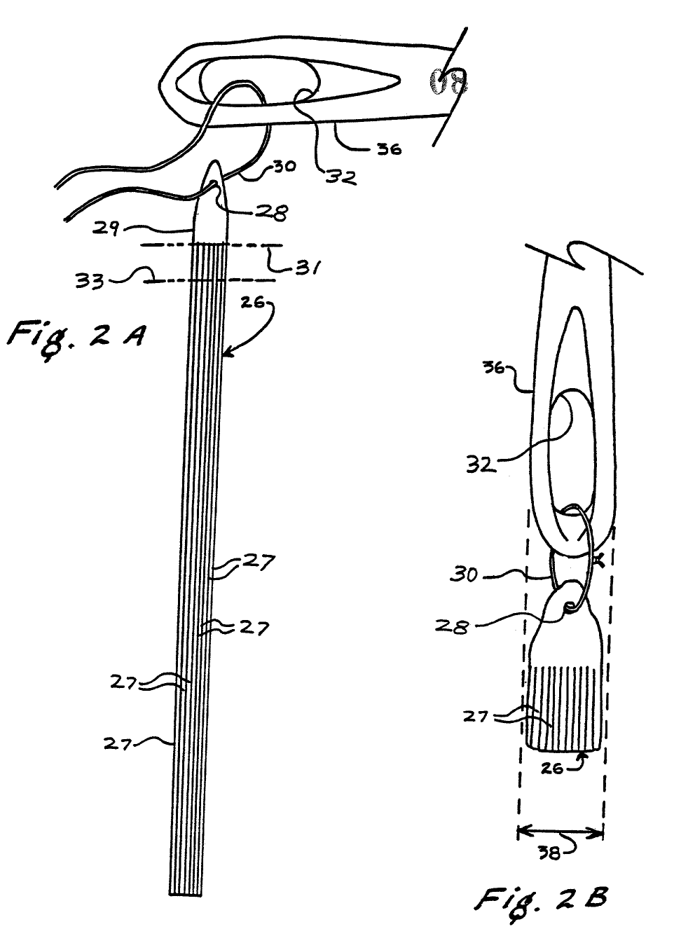

Referring to FIG. 1, a sheet 20 of biocompatible material such as GORE S.A.M. (Subcutaenous Augmentation Material ) (e.g., product No. 1SAM102, W. L. Gore & Associates, Inc. Flagstaff, Arizona) is fixtured onto a laser (not shown) and the laser is programmed to follow various cut patterns 22 and 24, to produce the prosthesis 26. Alternatively, the prosthesis 26 can be cut by steel rule die (not shown) or by any other suitable means. While the GORE S.A.M. is preferred, it is understood that many biocompatible materials may be suitable. As shown in FIG. 2A, the prosthesis 26 has a suture attachment feature 28, such as the eyelet shown, allowing rapid threading of a suture 30 through the suture attachment feature 28 and the eye 32 of the surgical needle 36. Because strands 27 are preferably formed by cutting through the sheet material to form the individual strands 27 without removing any material between adjacent strands, it is seen that a transverse cross section taken at transverse section line 31 through joined region 29 is of an area that is less than or equal to the transverse cross sectional areas of the multiplicity of strands 27 taken at transverse section line 33. The transverse cross sectional areas of the multiplicity of strands are the sum of the transverse cross sectional area of each of the individual strands 27 are formed by a method that involves the removal of material from between adjacent strands as suggested by FIG. 1, it is apparent that the transverse cross sectional area through the joined region 29 taken at transverse section line 31 will be significantly greater than the transverse cross sectional areas of the individual strands 27 taken at transverse section line 33.

As shown in FIG. 2B, the diameter of the prosthesis 26 may be equal to or less than the diameter 38 of needle 36 for minimal resistance during the process of pulling the prosthesis through living tissue.

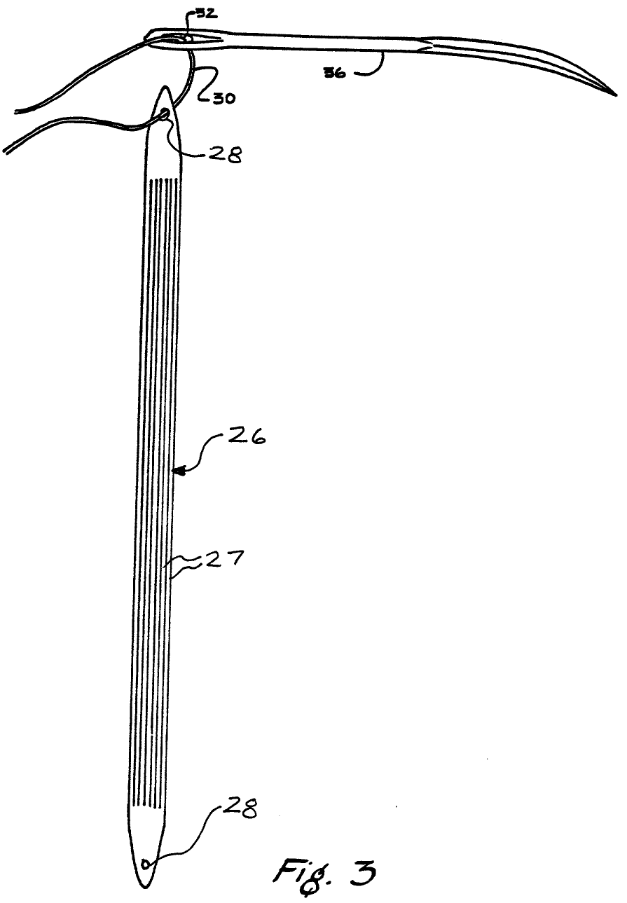

FIG. 3 describes a prosthesis 26 with attachment features 28 on both ends, allowing attachment of a needle 36 by passing a suture 30 through the eye 32 of the needle 36 and through the attachment feature 28. Thus the surgeon has the added flexibility to attach the needle to either end of the prosthesis

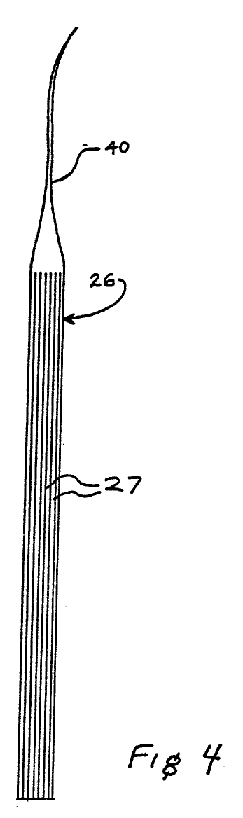

FIG. 4 shows a prosthesis 26 with an integral needle 40. The needle 40 can be formed out of PTFE and additional polymers, such as FEP or by densifying the porous PTFE in the region 40 using heat and/or pressure.

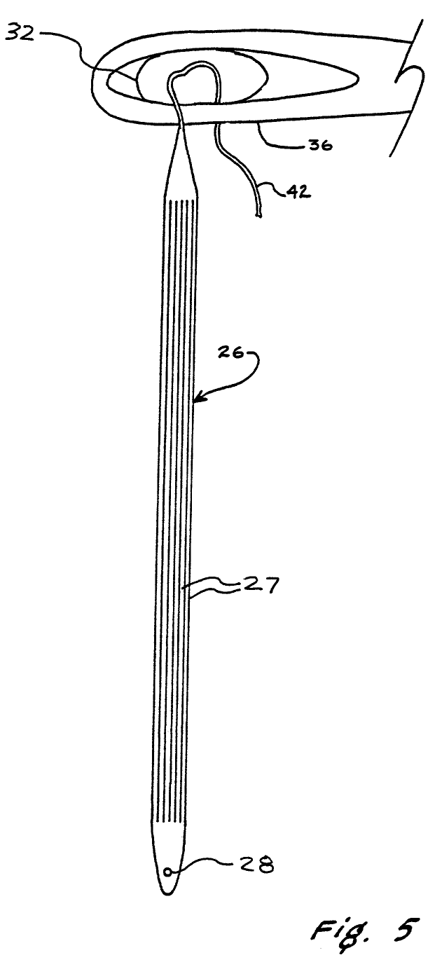

As shown in FIG. 5, the prosthesis 26 can have an integral threading means 42, such as the integrally joined suture shown, allowing rapid attachment to a needle 36, by feeding the threading means 42 through the needle eye 32 and securing the prosthesis 26 to the needle with a knot.

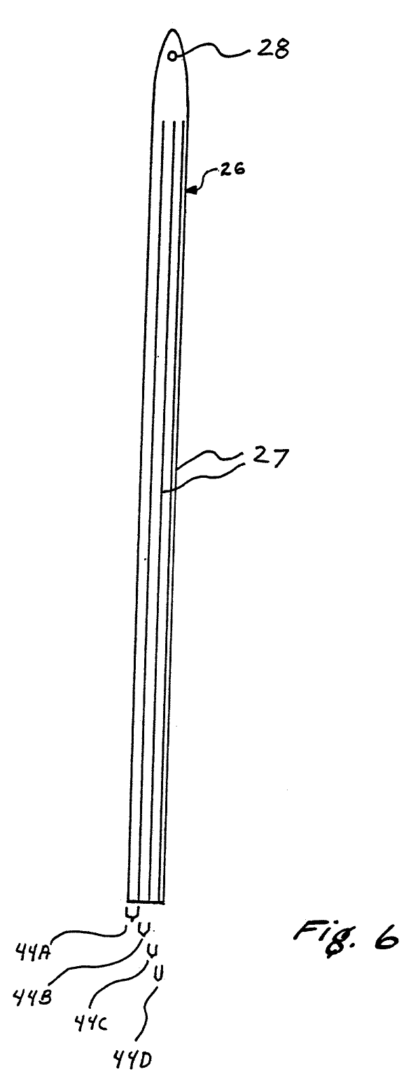

As shown in FIG. 6, the prosthesis 26 can have varying strand widths 44A-D. The varying strand widths allow precise and rapid tailoring of the augmentation.

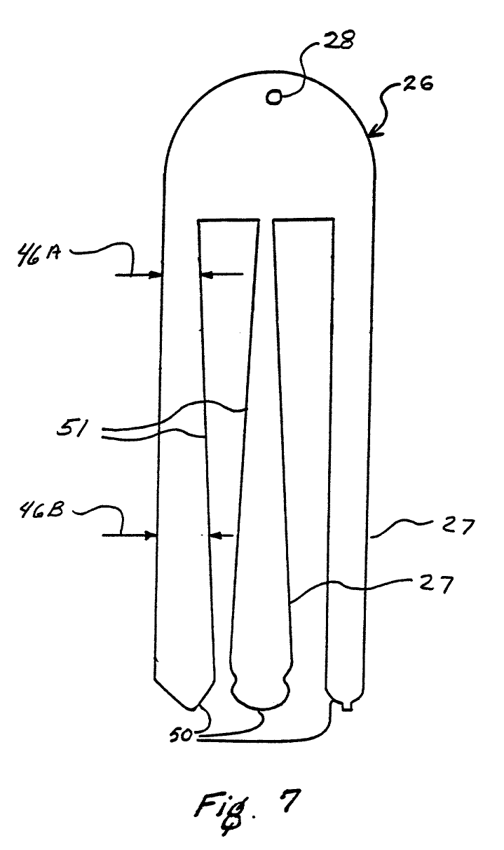

As shown in FIG. 7, the prosthesis 26 can have varying widths 46A and 46B along the length of an individual strand 27. In addition an individual strand 27 can have any shape at its end or termination 50. Further, the cut edges 51 of individual strands 27 can be non-parallel, making the prosthesis 26 wider or narrower at one end.

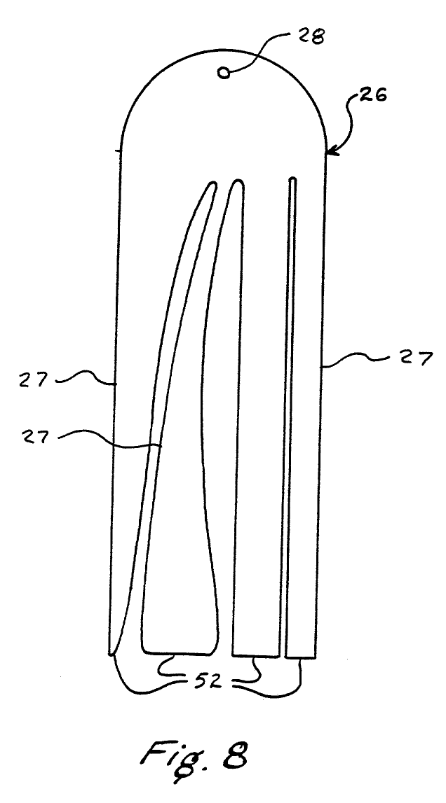

As shown in FIG. 8, the prosthesis 26 may be provided with varying shapes 52 along the length of individual strands 27.

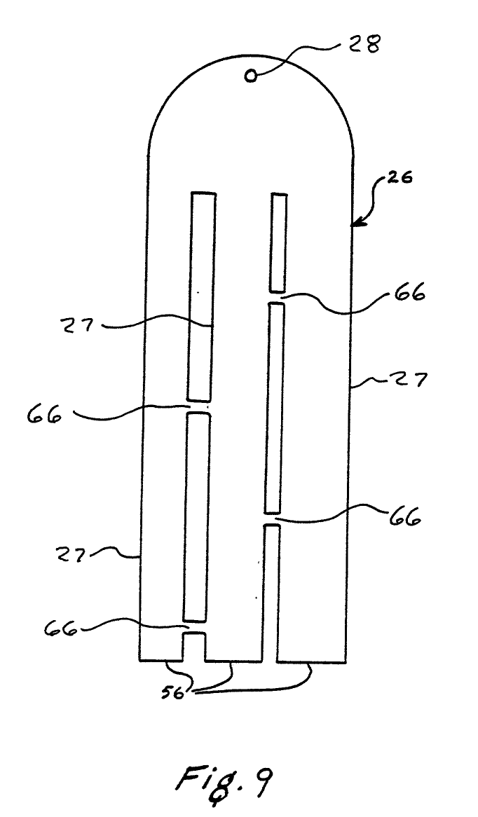

As shown in FIG. 9, the individual strands 27 of the prosthesis 26 can have cross connections 66 at various locations along the lengths of strands 27, which keep the individual strands 27 together after subcutaneous implantation. The prosthesis 26 may be provided with or without void spaces between adjacent strands as desired.

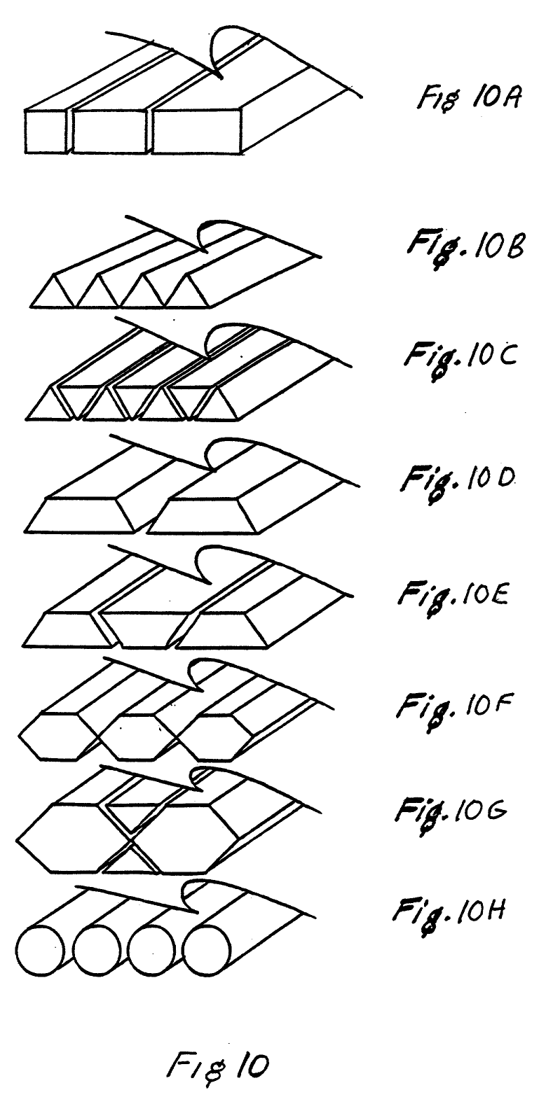

FIGS. 10A-H show a variety of transverse cross sectional configurations of the strands. These configurations can be manufactured by varying the cut patterns. For example in FIG. 10C, the triangles can be produced by changing the laser cut angle from zero degrees (perpendicular), to a sixty degree angle. Other configurations such as a circular shape shown in FIG. 10H, can be produced by a secondary operation following cutting of the strands. An example of a secondary operation would be drawing the cut strands through a forming die to alter the final cross sectional configuration.

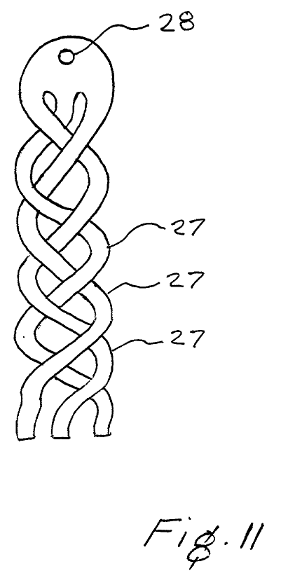

FIG. 11 shows a braided strand configuration. By braiding the strands prior to subcutaneous insertion, open areas or voids are created between the individual strands. These open areas between the strands will allow enhanced tissue ingrowth to occur throughout the array of strands. This ingrowth may strengthen the attachment of the device to surrounding tissue and reduce the risk of the device being extruded from the implant site.

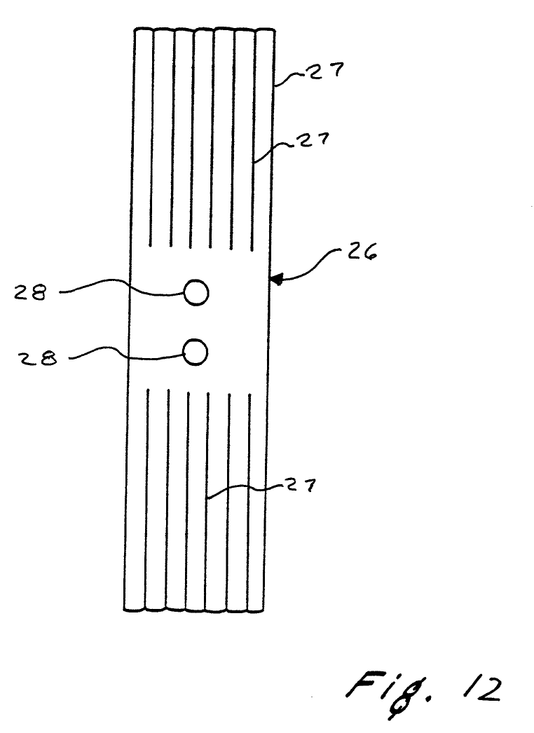

FIG. 12 shows an array of strands that are integrally joined in the center of the array. This array can be cut by the surgeon along the center of the array to form two separate, integrally joined arrays of strands. For filling more substantial voids, the array may be folded in half transversely between eyelets 28 allowing both halves of the uncut array to be tunneled below the dermis; the integrally joined section may then be cut off prior to closure of the incisions.

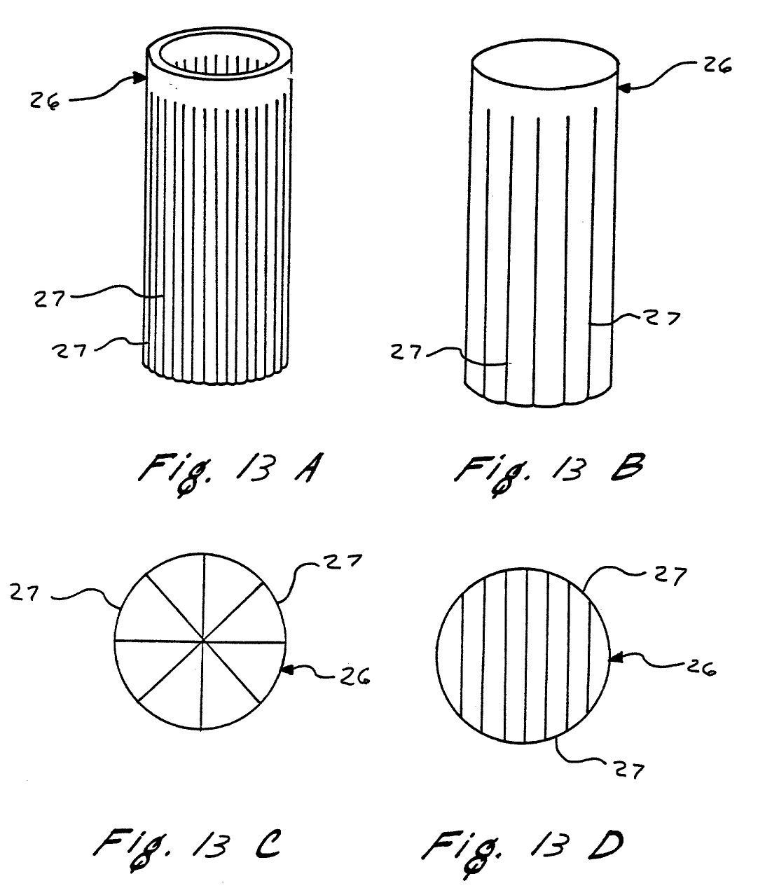

FIG. 13A shows a hollow tube, such as a vascular graft, cut into integrally joined strands. This device may be desirable for filling tissue void spaces of substantially circular cross sectional shapes. FIG. 13B describes an alternative wherein a solid rod is cut into integrally joined strands. This device will also conform to a substantially circular cross section when inserted below the dermis. FIGS. 13C and D show end views of the solid rod, with two possible strand cut patterns. These or other cut patterns can be used to form the integrally joined strands on either a solid rod or a hollow tube.

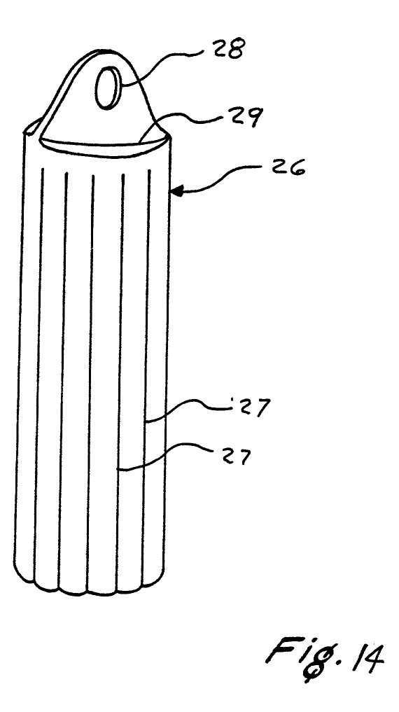

FIG. 14 shows a solid rod cut into integrally joined strands. The integrally joined region 29 of this device 26 also includes an attachment feature 28 to allow rapid attachment to a suture, surgical needle or other surgical instrument.

While the principles of the invention have been made clear in the illustrative embodiments set forth above, it will be obvious to those skilled in the art to make various modifications to the structure, arrangement, proportion, elements, materials and components used in the practice of the invention. To the extent that these various modifications do not depart from the spirit and scope of the appended claims, they are intended to be encompassed therein.

We claim:

1. An implantable tissue augmentation device comprising a multiplicity of strands of biocompatible material, each of said strands having at least one end wherein the multiplicity of strands are integrally joined at the at least one end of each of the multiplicity of strands.

2. An implantable tissue augmentation device according to claim 1 wherein the biocompatible material is selected from the group consisting of collagen, resorbable polymers, polyethylene terephthalate, polydimethyl siloxane, polypropylene, polyester, polyethylene, polyurethane and fluoropolymers.

3. An implantable tissue augmentation device according to claim 2 wherein the fluoropolymer is porous polytetrafluoroethylene.

4. An implantable tissue augmentation device according to claim 1 wherein the multiplicity of strands are integrally joined at a region containing an eyelet.

5. An implantable tissue augmentation device according to claim 1 wherein each of said strands has two ends wherein the multiplicity of strands are integrally joined at the two ends.

6. An implantable tissue augmentation device according to claim 1 wherein the multiplicity of strands are integrally joined at a region which includes an integrally joined suture.

7. An implantable tissue augmentation device according to claim 1 wherein each of said strands has a length and a width and further wherein the width of a first strand differs from the width of a second strand.

8. An implantable tissue augmentation device according to claim 1 wherein each of said strands has a length and a width and further wherein the width of a strand varies along the length of that strand.

9. An implantable tissue augmentation device according to claim 1 wherein at least one strand has a polygonal cross section.

10. An implantable tissue augmentation device according to claim 9 wherein at least one strand has a triangular cross section.

11. An implantable tissue augmentation device according to claim 9 wherein at least one strand has a rectangular cross section.

12. An implantable tissue augmentation device according to claim 9 wherein at least one strand has a hexagonal cross section.

13. An implantable tissue augmentation device according to claim 9 wherein at least one strand has a trapezoidal cross section.

14. An implantable tissue augmentation device according to claim 1 wherein at least one strand has a round cross section.

15. An implantable tissue augmentation device according to claim 1 wherein the strands have a length and wherein at least two strands are integrally joined at intervals along their length.

16. An implantable tissue augmentation device according to claim 1 wherein the multiplicity of strands are braided together.

17. An implantable tissue augmentation device according to claim 1 wherein the multiplicity of strands have a transverse cross sectional area and wherein the multiplicity of strands are joined at a joined region wherein the joined region has a transverse cross sectional area that is less than or equal to the transverse cross sectional area of the multiplicity of strands.
